(19) United States
(12) Patent Application Publication
(10) Pub. No.: US 2017/0156769 A1
(43) Pub. Date: Jun. 8, 2017

(54) BONE PLATES CONFIGURED TO RIGIDLY FIX FRAGMENTS OF A BONE TO ONE ANOTHER

(71) Applicant: Carl Pasquale Giordano, New Vernon, NJ (US)

(72) Inventor: Carl Pasquale Giordano, New Vernon, NJ (US)

(21) Appl. No.: 15/417,544

(22) Filed: Jan. 27, 2017

Related U.S. Application Data
(63) Continuation-in-part of application No. 13/438,912, filed on Apr. 4, 2012, now Pat. No. 9,615,860.

(51) Int. Cl. A61B 17/80; A61B 17/70; A61B 17/86

(52) U.S. Cl. CPC A61B 17/8052; A61B 17/8061; A61B 17/86; A61B 17/7059

(57) ABSTRACT

Bone plates for rigidly fixing fragments of a bone to one another are provided. In certain aspects, the apparatus is a bone plate that has an outer edge that conforms to a shape of a pedicle and/or lamina of the vertebra or a long bone, and an inner section that facilitates access to the fractured area when the plate is secured to the bone.

### CROSS REFERENCE TO RELATED APPLICATIONS

This application is a continuation-in-part of U.S. patent application Ser. No.: 13/438912, filed on Apr. 4, 2012, the entirety of which is incorporated by reference herein.

### BACKGROUND

The Background of the Invention is described, by way of example only, as applying to fixing a fracture of a pars interarticularis. However, aspects of the invention disclosed herein may be applied to any type of bone fracture.

Spondylolysis is a condition in which a crack or fracture occurs in rear portions of vertebrae of a patient's spinal column. The spinal column includes a ring of bone located at the middle and posterior portion of the spinal column that protects the spinal cord within the spinal column. The specific area that becomes cracked or fractured is known as the pars interarticularis, or pars, and is the area between a pedicle and lamina of a vertebra.

The crack or fracture, often known as a “pars fracture” or “pars defect,” is generally caused by excessive or repeated strain to the pars area, although congenital effects may also cause a pars fracture. Spondylolysis commonly occurs in the lower back at lumbar vertebra number five (L5). For example, Spondyloysis may affect athletes that repeatedly bend backwards, such as athletes in gymnastics, karate, and/or football.

Under strain, a vertebra will initially add bone cells around an injured area. However, once the strain is too great, or too violent, a stress fracture will form in the vertebra. Such a fracture will often occur within the pars, and multiple and/or persistent fractures lead to a pars defect.

In some cases, a pars defect will occur on two sides of a vertebra. When this occurs, a vertebra may translate or move towards or away from neighboring vertebrae, leading to a condition know as Spondylolisthesis.

In order to illustrate where Spondyloysis occurs, FIGS. 1A-1B depict views of a pars fracture or defect within a vertebra. FIG. 1 A illustrates a side view 100 of vertebrae of a spinal column. Vertebra 110 is separated from vertebra 120 by a disk 115. Vertebra 110 is a healthy vertebra, with a pedicle 112 and lamina 114 surrounding a pars area. In contrast, vertebra 120 in an unhealthy or affected vertebra, with a pedicle 122 and lamina 124 surrounding a pars fracture or defect 125. That is, the pars fracture 125 occurs in vertebra 120 between a pedicle 122 and lamina 124 of the vertebra.

FIG. 1B depicts the pars defect from another view, namely from a top view 130 of the vertebra 120. The pars fracture 125 is located between the pedicle 122 and the lamina 124, and may cause separation of the lamina 124 from the pedicle 122. Once separated, or partially separated, the lamina 124 may translate or move relative to the pedicle 122, causing pain and/or other problems.

Conventional treatment of Spondyloysis, Spondylolisthesis, and other conditions due to fractures in the pars interarticularis of a vertebra include (1) the fusing together of vertebral segments surrounding a pars fracture or (2) the attempting to repair the pars fracture. However, there are disadvantages with conventional treatments.

Fusing vertebral segments together often leads to a loss of motion across an affected area, which may cause additional stress to surrounding vertebrae and accelerate arthritis and other conditions within the vertebrae.

Conventional repair procedures, such as using wires or screws to hold vertebral fragments together, suffer from high failure rates, because the procedures often do not maintain fractured vertebral fragments in proper position during a bone grafting or other treatments.

In some cases, bone plates are used during surgical repairs within the spine. For example, a first reference, U.S. Published Patent Application No. 2010/0082067, to Kondrashov, entitled “System and Method to Stabilize a Spinal Column Including a Spinolaminar Locking Plate,” discloses an anchoring plate adapted to stabilize a spinal column. The anchoring plate may have a shape that conforms to a lamina of a targeted vertebra, but is used and configured in order to stabilize a spinal column and reduce compression of spinal nerves.

As another example, a second reference, U.S. Published Patent Application No. 2005/0119657, to Goldsmith, entitled “Facet Triangle Spinal Fixation Device and Method of Use,” discloses a device adapted to bilaterally secure two vertebrae together.

In both examples, the disclosed bone plates are generally utilized to stabilize a spinal column and proximate vertebrae within a spinal column.

While aforementioned medical devices and treatments are generally suitable for a particular purpose, such devices are not sufficiently suitable for the purpose discussed in the present invention. Thus, it is clear that there exists a need in the art for a treatment device that overcomes these problems and progresses the state of the art, as well as one that provides additional benefits enumerated in the present application.

### SUMMARY OF THE INVENTION

In view of the foregoing disadvantages inherent in the art, in accordance with one aspect of the present invention, an apparatus configured and/or adapted to attach to a pars area of a vertebra is described. In addition, an apparatus configured to attach to a long bone is also disclosed. Long bones include bones in the lower limbs namely the tibia, fibula, femur, metatarsals, and phalanges and bones in the upper limbs namely the humerus, radius, ulna, metacarpals, and phalanges. In some aspects of the present invention, a bone plate is formed of a shape similar to a shape of a pedicle and/or lamina of the vertebra and/or long bone.

In some aspects of the invention all embodiments disclosed herein include an outer edge of an elongated peripheral bone plate that conforms to the contour of an outer edge of a pedicle, lamina or long bone. In other aspects of the invention, all embodiments of a bone plate disclosed herein may have a lower bone-contacting surface that conforms to a two-dimensional or three-dimensional geometry of the bone to which it is affixed.

In some aspects, the bone plates in accordance with the invention disclosed herein are made from materials that are sufficiently rigid to hold the fractured bones in place but malleable enough that the plate can be deformed by the surgeon immediately prior to placement in a patient in order to conform it to the particular anatomy of the patient.

That is, in some aspects, the bone plate may be configured to include an anatomical geometry similar to a geometry of an anatomical region in which the plate is attached during rigid fixation of a pars fracture or fracture of a long bone. The geometry, or shape, may be similar to a geometry of a pedicle, a geometry of a lamina, a geometry of both the pedicle and the lamina surrounding a pars fracture or a geometry of a long bone.

As mentioned, it is contemplated by the present disclosure that the bone plate may include an outer section that generally conforms to a shape of an attachment region, such as a pedicle and/or lamina, and an inner section that facilitates access to a fractured area when the bone plate is attached to a vertebra.

Furthermore, it is contemplated by the present disclosure that the bone plate includes or be part of a support structure, and may include a fragment fixation section and a support section, whereby the bone plate provides for the rigid fixation of vertebral fragments and for the support of an affected vertebra.

Thus, in certain embodiments, the plate is configured and/or adapted to facilitate attachment to a pars area of a vertebra to provide rigid fixation of the bones during bone grafting and subsequent healing of a fracture within the pars area, while providing other inherent benefits.

In certain aspects of the invention a bone plate is configured to be attached to a single pedicle and a single lamina defining a single fractured pars area of a vertebra. The plate is a deformable or non-deformable monolithic or non-monolithic structure having a shape similar to a shape of a single pedicle and a single lamina defining the fractured pars area of the vertebra. The bone plate is defined in one aspect by a continuous, unbroken elongate peripheral external rim and a continuous, unbroken elongate peripheral internal rim forming a closed or open loop. In other aspects the bone plate is configured in two portions that may be interlockingly joined together. In other aspects the bone plate is substantially C-shaped in cross section. The external and internal rims of the bone plate are generally irregular along their entirety. The bone plate has an elongate upper surface and an opposing elongate lower bone contacting surface, said upper and lower elongate surfaces connecting said elongate external rim to said elongate internal rim and spacing the elongate rims from one another by an irregular distance of said surfaces.

The external rim is structured to conform to a contour of a single pedicle and a single lamina. The lower bone contacting surface of the bone plate may be shaped to conform to a three dimensional geometry of a single pedicle and a single lamina.

The upper surface and the lower bone contacting surface of the pedicle plate section may lack screw holes therethrough. The upper surface and the lower bone contacting surface of the lamina plate section include screw holes extending therethrough. In one aspect the lamina plate section may be configured to accommodate a crankshaft screw that rotatably advances a hook in the lamina edge or through the lamina.

The internal rim of the pedicle plate section defines a pedicle screw receiving hole having a crescent shape bounded by the inner rim of the pedicle plate section and opening into a non-expandable narrow gap defined between opposing portions of the internal rim that is in fluid communication with a lamina open area encircled by the internal and external rims of the lamina plate section. In other aspects, the internal rim of the pedicle plate section defines a pedicle screw receiving hole that does not open into a non-expandable narrow gap but is in fluid communication with an open area defined by the inner rim of the lamina plate portion. When implanted the bone plate is structured to overlie a fractured pars area.

Generally, a total surface area between the external rim and the internal rim of the lamina plate portion may be less than a total surface area of the lamina open area. Generally, a total surface area between the opposing external rims of the pedicle plate section may be smaller than a total surface area between opposing edges of the external rim of the lamina plate section. A dimension of the narrow through gap is smaller than a dimension of the pedicle screw receiving hole which is smaller than a dimension of the lamina open area.

In other aspects of the invention, a substantially rectangularly shaped elongate bone plate is provided. The rectangularly shaped elongate bone plate includes an outer rim configured to conform to the edge of the bone to which it is attached and an inner rim defining an open area. The elongate bone plate has an upper surface and a bone contacting surface, the bone contacting surface configured to conform to the two-dimensional or three-dimensional geometry of the bone to which it is affixed. The elongate bone plate may include a plurality of openings through the upper and bone contacting surfaces and are adapted to receive a screw or other fastener to affix the bone plate to a long bone. The plate is configured to rigidly fix fragments of a long bone together while the open area defined by the inner rim provides access to the surgeon during the healing process to apply bone graft materials and the like.

By using the bone plates described herein, the success of repairing pars fractures, pars defects and long bone fractures may be increased with respect to conventional approaches or treatment techniques.

Thus, there has been summarized and outlined, generally in broad form, a plurality of the most important features of the present invention. While this summary is presented so that the novelty of the present contribution to the related art may be better appreciated, it will further be apparent that additional features of the invention described hereinafter (which will form the subject matter of the claims appended hereto) will further define the scope, novelty, and in certain instances the improvements upon any existing art. The following description provides specific details for a thorough understanding of, and enabling description for, various examples of the technology. One skilled in the art will understand that the technology may be practiced without many of these details and it is to be readily understood that the invention presented herein is not limited in its application to the details of construction and to the arrangements of the components set forth in the following description or illustrated in the various figures integrated and categorized herein. For example, in some instances, well-known structures and functions have not been shown or described in detail to avoid unnecessarily obscuring the description of the examples of the technology. It is intended that the terminology used in the description presented below be interpreted in its broadest reasonable manner, even though it is being used in conjunction with a detailed description of certain examples of the technology. Although certain terms may be emphasized below, any terminology intended to be interpreted in any restricted manner will be overtly and specifically defined as such in this Detailed Description section. Those skilled in the art will appreciate that the disclosure of the present invention may readily be utilized as a basis for the designing of other similar structures, methods and systems for carrying out the various purposes and objectives of the present invention. Thus, the claims as set forth shall allow for such equivalent constructions insofar as they do not depart from the spirit and scope of the present invention as described herein.

### BRIEF DESCRIPTION OF THE FIGURES

A further understanding of the present invention and the objectives other than those set forth above can be obtained by reference to the various embodiments set forth in the illustrations of the accompanying figures. Although the illustrated embodiments are merely exemplary of the present invention, apparatus and method of use of the invention, in general, together with further objectives and advantages thereof, may be more easily understood by reference to the drawings, examples, and the following description. The examples and figures are not intended to limit the scope of this invention, which is set forth with particularity in the claims as appended or as subsequently amended, but merely to clarify and exemplify the invention. The detailed description makes reference to the accompanying figures wherein:

FIGS. 1A-1B are perspective views of common pars interarticularis areas having pars defects.

FIG. 2 is a perspective view of a plate configured to be attached to a pars area of a vertebra.

FIG. 3 is a perspective view of a plate attached to a pars area of a vertebra.

FIG. 4 is a flow diagram illustrating a routine for developing a plate used to rigidly fix a pedicle to a lamina.

FIGS. 5A-5C are perspective views of various configurations of bone plates configured to be attached to a pars area of a vertebra.

FIG. 6 is a perspective view of a bone plate that includes a section configured to be attached to a pars area of a vertebra.

FIG. 7 is a perspective view of a bone plate that includes a lamina plate section that interlocks with a pedicle plate section.

FIG. 8 is a perspective view of a bone plate that includes a crankshaft screw that advances a hook into a lamina.

FIGS. 9 and 10 are perspective views of a bone plate that may be utilized to fix long bone fractures.

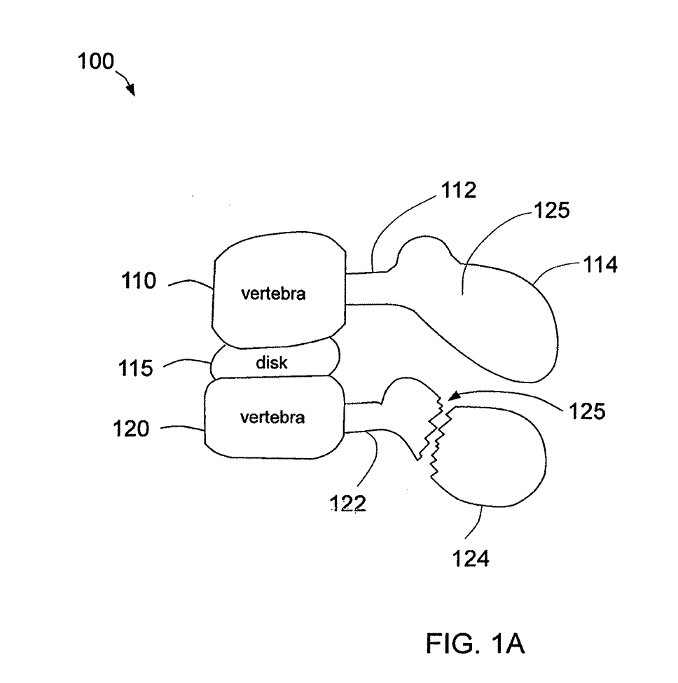

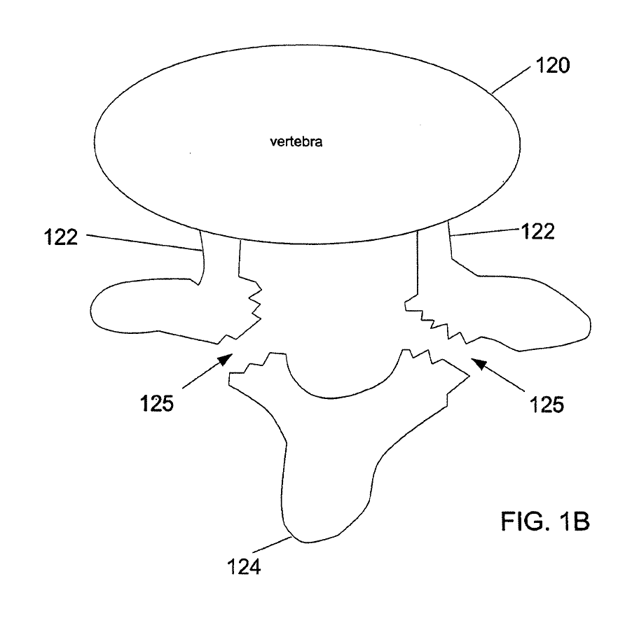

### DETAILED DESCRIPTION OF THE PRESENT INVENTION

A detailed illustrative embodiment of the present invention is disclosed herein. However, techniques of manufacture and resulting structures in accordance with the present invention may be embodied in a wide variety of forms and modes, some of which may be quite different from those in the disclosed embodiment. Consequently, the specific structural details disclosed herein are merely representative, yet in that regard, they are deemed to afford the best embodiment for purposes of disclosure and to provide a basis for the claims herein which define the scope of the present invention. The following presents a detailed description of several examples of the present invention.

Moreover, well known methods, procedures, and substances for both carrying out the objectives of the present invention and illustrating the preferred embodiment are incorporated herein but have not been described in detail as not to unnecessarily obscure novel aspects of the present invention.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The apparatus or device described herein (e.g., “pars bone plate”) is a novel device configured to attach to a vertebra to assist during healing of a pars fracture of the vertebra. The pars plate may be formed in a shape similar to the vertebra or section of the vertebra in order to facilitate attachment and use within a compact and complexly structured area of the spinal column, and so on. Such a device may provide an alternative path for the treatment of pars fractures, among other benefits.

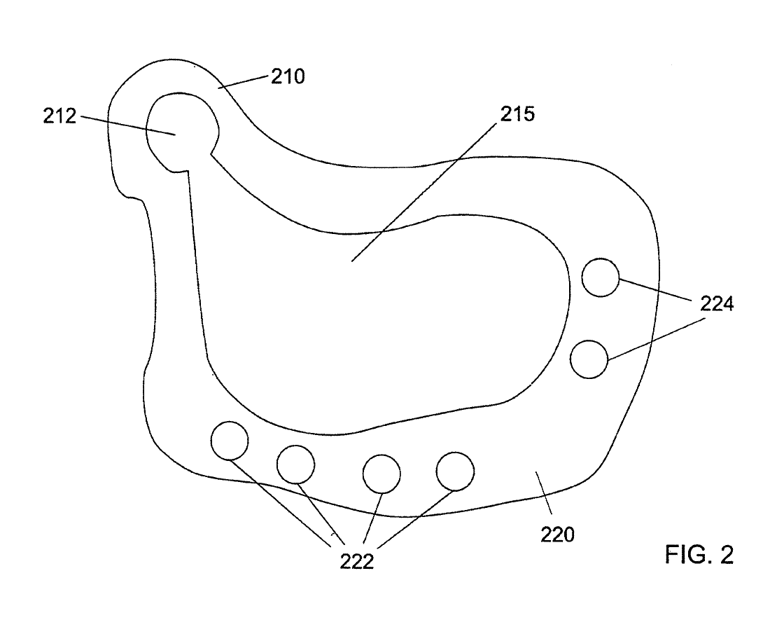

Turning to FIG. 2, provided is a perspective view of a bone plate configured to be attached to a pars area of a vertebra. The bone plate 200 includes a pedicle section 210 having a pedicle attachment hole 212 and a lamina section 220 having lamina attachment holes 222 and spinous process attachment holes 224. The attachment holes 212, 222, 224 may be configured to receive screws or other attachment devices that attach the bone plate 200 to a vertebra.

The bone plate 200 also includes an open section 215, which provides access to a fracture when the bone plate is attached to a pars area. The open section 215 may be configured as shown in the Figure, or may include an opening of various sizes and/or shapes, depending on the size and/or shape of the vertebra, the size and/or shape of the affected area of the vertebra, the size and/or shape of a fracture within the pars area, and so on.

The pedicle section 210 and lamina section 220 of the bone plate 200 may be formed having an I-beam cross section, and are adapted to conform to the shape of the pars area in which the bone plate 200 is attached. The shape of the bone plate 200 will now be discussed with respect to the shape of a vertebra in which the bone plate 200 is attached.

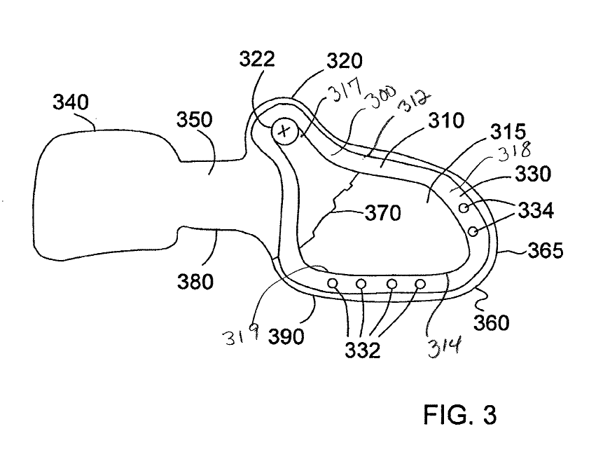

FIG. 3 is a perspective view 300 of a bone plate attached to a pars area of a vertebra 340. The vertebra 340 includes a pedicle 350 and a lamina 360, which includes a spinous, or transverse, process 365. The depicted vertebra has a fracture 370 in the pars area between the pedicle 350 and the lamina 360. When fractured, the vertebra 340 is fragmented into a vertebral fragment 380 that includes the pedicle 350, and a vertebral fragment 390 that includes the lamina 360.

A bone plate 310 used for rigid fixation of the fracture 370 is attached to the vertebra in order to rigidly fix the vertebral fragment 380 containing the pedicle 350 to the vertebral fragment 390 containing the lamina 360 in order to facilitate successful healing of the fracture 370.

The bone plate 300 comprises a monolithic, rigid elongate peripheral rim 310 having a pedicle section 320 and a lamina section 330. The bone plate 300 includes an upper surface 318 and a lower bone-contacting surface 319. The lower bone-contacting surface 319 may conform to a two- or three-dimensional geometry of the pedicle and lamina. The elongate peripheral rim 310 has an outer edge 312 that conforms to the contour and shape of the pedicle 350. An inner edge 314 defines a screw receiving opening 316 that enables a screw 322 to fix the pedicle section 320 of the bone plate 300 to the pedicle 350. Additionally, in some examples the bone plate 300 may be attached to the vertebral fragment 380 at various different areas of the vertebral fragment 380 in addition to the manner depicted.

As shown, the pedicle section 320 of the bone plate 310 is shaped and/or is formed having a geometry that may be similar to a shape and/or anatomical geometry of the pedicle 350. Shaping the pedicle section 320 in such a manner enables the bone plate 310 to conform to the area in which it is fixed, which allows for the bone plate to be placed within such a dense, complex area of a spinal column, among other benefits.

The bone plate 310 also includes a lamina section 330 that may be of a shape similar to a shape and/or anatomical geometry of the lamina 360. The lamina section 330 of the bone plate 310 includes one or more attachment sections, includes attachment sections that facilitate using lamina screws 332 to attach the bone plate 310 to the lamina 360, attachment sections that facilitate using spinous process screws 334 to attach the bone plate 310 to the spinous process 365, and so on. Additionally, in some examples the bone plate 310 may be attached to the vertebral fragment 390 at various different areas of the vertebral fragment 390 in addition to the manner depicted.

As shown, the lamina section 330 of the bone plate 310 is shaped and/or may be formed having a geometry that is similar to a shape and/or geometry of the lamina 360 and/or spinous process 365. Shaping the lamina section 330 in such a manner enables the bone plate 310 to conform to the area in which it is fixed, which allows for the bone plate to be placed within such a dense, complex area of a spinal column, among other benefits.

In certain embodiments, the bone plate 310 includes an opening 315 or inner portion that provides access to the fracture 370 within the pars area of the vertebra 340. That is, the bone plate 310 may be configured to give surgeons access to a fracture 370 in order to perform bone grafting procedures while the bone plate 310 rigidly maintains the vertebral fragments together, may be configured to allow for local vascular in-growth of the fracture 370, may be configured to enable access of the fracture 370 for post operation CT Scan imaging procedures, and so on. Those of skill in the art will appreciate that opening 315 is in fluid communication with screw receiving opening 317.

Thus, in certain embodiments, the bone plate 310 includes an outer portion that is shaped similar to areas of a vertebra in which the bone plate attaches and is shaped similar to the areas of the vertebra in which the bone plate attaches, and an inner portion that enables access to a fractured or repaired area of the vertebra.

Determining the Size and/or Shape of the Pars Bone Plate

In addition to conforming to the shape of the attachment areas of a vertebra, the bone plate, in certain embodiments, may also be formed of a size that conforms to the size of the attachment areas. In some cases, bone plates of differing sizes, such as sizes based on anatomical averages of sizes of the vertebra, may be formed. A left bone plate may be formed, a right bone plate may be formed, a bilateral bone plate may be formed, and so on. However, in some cases, a more specific sized and/or shaped bone plate may be required.

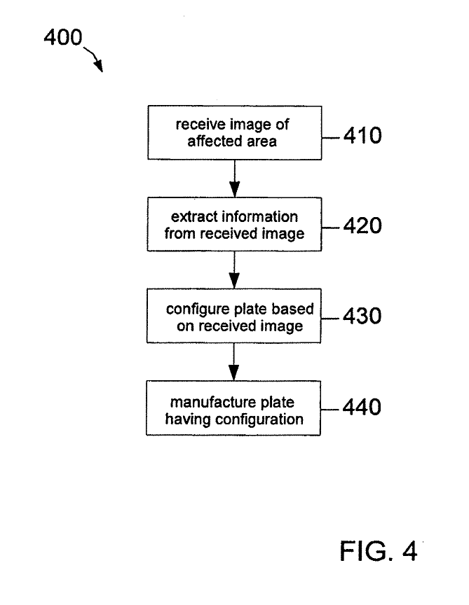

In certain embodiments, the size and shape of a bone plate may be determined based on images or other information associated with the area in which the bone plate is to be attached. FIG. 4 is a flow diagram illustrating a routine 400 for configuring and manufacturing a bone plate used to rigidly fix a pedicle to a lamina.

FIG. 4 and the following discussion provide a brief, general description of a suitable computing environment in which a bone plate configuration system can be implemented. Although not required, aspects of the system are described in the general context of computer-executable instructions, such as routines executed by a general-purpose computer, e.g., a server computer, wireless device, or personal computer. Those skilled in the relevant art will appreciate that the system can be practiced with other communications, data processing, or computer system configurations, including: Internet appliances, network PCs, mini-computers, mainframe computers, medical computing devices, and the like. Indeed, the terms “computer” and “computing system” are generally used interchangeably herein, and refer to any of the above devices and systems, as well as any data processor.

Aspects of the system can be embodied in a special purpose computer or data processor that is specifically programmed, configured, or constructed to perform one or more of the computer-executable instructions or routines explained in detail herein. Aspects of the system can also be practiced in distributed computing environments where tasks or modules are performed by remote processing devices, which are linked through a communications network, such as a Local Area Network (LAN), Wide Area Network (WAN), Storage Area Network (SAN), Fibre Channel, or the Internet. In a distributed computing environment, program modules may be located in both local and remote memory storage devices.

Aspects of the system may be stored or distributed on computer-readable media, including magnetically or optically readable computer discs, hard-wired or preprogrammed chips (e.g., EEPROM semiconductor chips), nanotechnology memory, biological memory, or other tangible data storage media. Indeed, computer implemented instructions, data structures, screen displays, and other data under aspects of the system may be distributed over the Internet or over other networks (including wireless networks), on a propagated signal on a propagation medium (e.g., an electromagnetic wave(s), a sound wave, etc.) over a period of time, or they may be provided on any analog or digital network (packet switched, circuit switched, or other scheme). Those skilled in the relevant art will recognize that portions of the system reside on a server computer, while corresponding portions reside on a client computer, and thus, while certain hardware platforms are described herein, aspects of the system are equally applicable to nodes on a network.

In step 410, a system, such as a bone plate configuration system, receives an image or images of an affected area of a vertebra. The received images may be two-dimensional and/or three-dimensional images, providing information about images areas in all dimensions.

For example, the image may be a partial or full image of a vertebra, a partial or full image of a lamina area, a partial or full image of a pedicle area, a partial or full image of a fracture area, a partial or full image of a pars area, and so on. The image may be taken using a number of different imaging techniques, such as radiography (e.g., x-rays), X-Ray computed tomography (e.g. CT Scans), and so on.

In step 420, the system extracts information from the received image or images. For example, the system may extract information associated with sizes of areas of an affected vertebra, such as sizes of the vertebra, sizes of a pedicle, sizes of a lamina, sizes of a spinous process, sizes of the fractures area, sizes of the pars area, sizes associated with distances between the vertebra and other surrounding vertebrae, and so on. The system may extract other information, such as information associated with a shape of various areas of the vertebra, information associated with a bone density of various areas of the vertebra, and so on.

In step 430, the system configures a bone plate to be attached to the vertebra based on the extracted information. For example, the system may generate a schematic of a bone plate based on the size and/or shape information extracted from the received images.

In step 440, the system manufactures a bone plate according to the determined configuration. For example, the system manufactures a bone plate that is based on the generated schematic. Thus, the system may be utilized to form bone plates that are optimizes in size and/or shape to the area in which they are to be attached. Such optimization may facilitate the use of bone plates in virtually every patient that suffers from Spondylolysis, such as very large or small patients, patients that have abnormal spinal column geometries, patients that have abnormal vertebra geometries, and so on.

Thus, in certain embodiments, the bone plates described herein further and expand upon the state of the art by providing devices that rigidly fix vertebral fragments together, improving over the conventional practice of using wires or other unsuitable fixation practices.

### Other Bone Plate Configurations

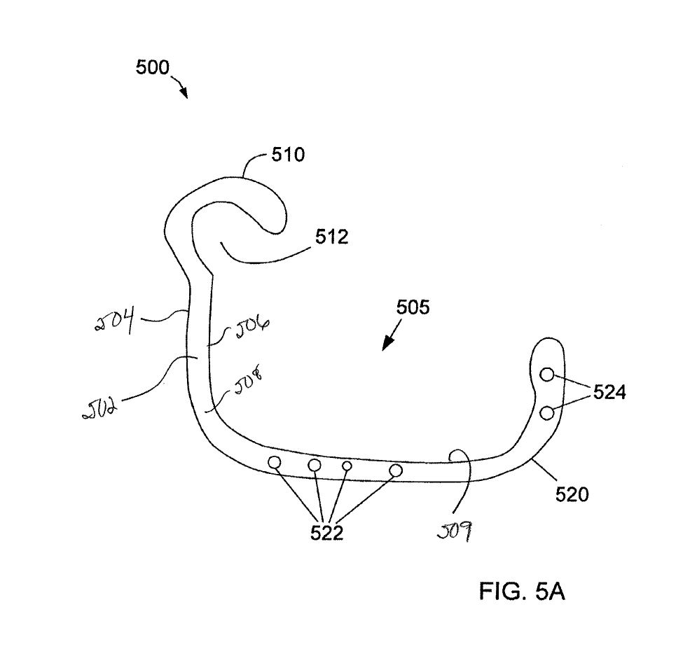

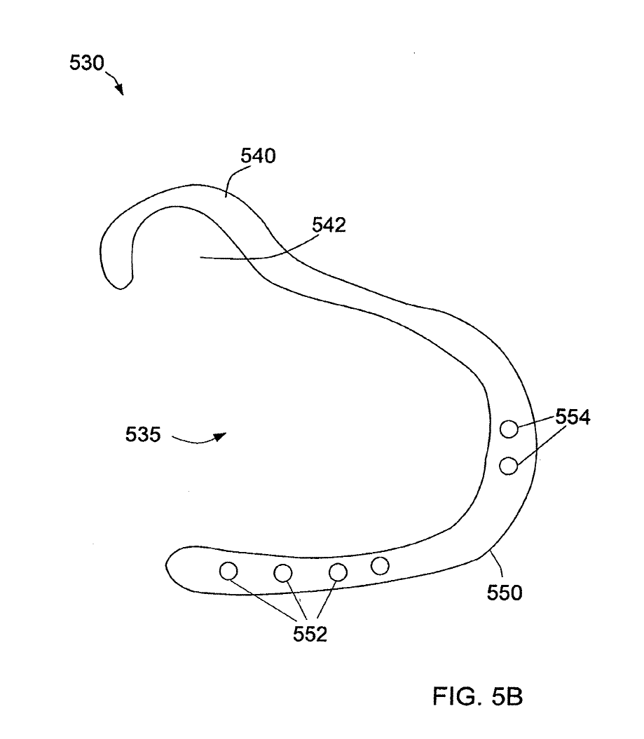

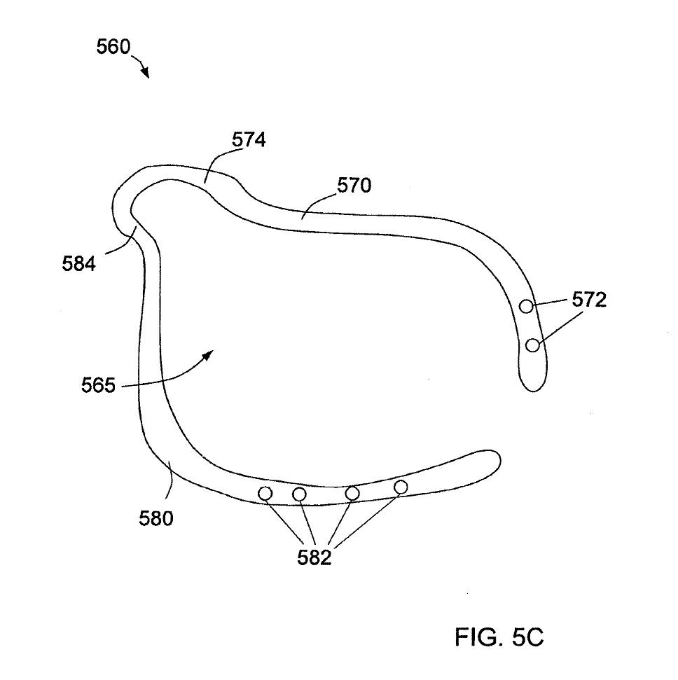

In alternative embodiments, the pars bone plate, such as bone plates 200 and 310, may be formed in a variety of different configurations. FIGS. 5A-5C are perspective views of bone plates to be attached to a pars area of a vertebra and are all generally C-shaped in cross section with the dimension of the open portion of the C varying.

FIG. 5A depicts a bone plate 500 comprising a monolithic, elongate peripheral rim 502. Peripheral rim 502 includes an outer edge 504 and an inner edge 506. Peripheral rim 502 includes a pedicle portion 510 and a lamina section 520. Lamina section 520 is configured to be positioned on a bottom portion of a lamina with the outer edge 504 conforming the contour of the lamina bottom portion. Peripheral rim 502 includes an upper surface 508 and a lower bone-contacting surface 509. Lower bone-contacting surface 509 may conform to a two- or three-dimensional geometry of the pedicle and lamina bottom portion. The inner edge 506 of the pedicle section 510 defines a hole 512 that facilitates use of a screw to attach the bone plate 500 to a pedicle of a vertebra. The lamina section 520 may include one or more lamina holes 522 and spinous process holes 524 that facilitate use of screws to attach the bone plate 500 to a lamina of a vertebra. The inner edge 506 of the lamina section 520 is configured to provide an opening 505 or access area, which enables a fracture to be accessible when the bone plate 500 is attached to a vertebra.

FIG. 5B depicts a configuration in which a bone plate 530 is configured to run along a top portion of a lamina. The bone plate 530 includes a pedicle section 540, a lamina section 550, and an opening 535 or inner section. The pedicle section 540 includes or is shaped to include a hole 542 that facilitates use of a screw to attach the bone plate 530 to a pedicle of a vertebra. The lamina section includes lamina holes 552 and spinous process holes 554 that facilitate use of screws to attach the bone plate 530 to a lamina of a vertebra. The bone plate 530 is configured to provide an opening 535 or access area, which enables a fracture to be accessible when the bone plate 530 is attached to a vertebra.

FIG. 5C depicts a configuration in which a bone plate 560 is formed of two pieces, one formed to run along a bottom portion of a lamina, and one formed to run along a top portion of the lamina. The bone plate 560 includes an upper piece 570 having a pedicle section 574 that includes or is shaped to include an attachment point that facilitates use of a screw to attach the upper piece 570 to a pedicle of a vertebra, and a lamina section that includes lamina holes 572 that facilitate use of screws to attach the upper piece 570 to a lamina of a vertebra.

The bone plate 560 also includes a lower piece 580 having a pedicle section 584 that includes or is shaped to include an attachment point that facilitates use of a screw to attach the lower piece 580 to a pedicle of a vertebra, and a lamina section that includes lamina holes 582 that facilitate use of screws to attach the lower piece 580 to a lamina of a vertebra.

The bone plate 560 is configured to provide an opening 565 or access area, which enables a fracture to be accessible when the bone plate 560 is attached to a vertebra.

One of ordinary skill in the art will appreciate that other configurations may be utilized when forming bone plates to be attached to rigidly fix vertebral fragment together when repairing pars fractures. For example, bone plates having multiple (e.g., two or more) pieces may be formed, bone plates having various cross-sectional configurations may be formed, bone plates having various internal opening layouts may be formed, bone plates configure to treat dual fractures may be formed, and so on.

In certain embodiments, a bone plate may be part of an integrated support device within the spinal column. That is, the bone plate may be configured to attach to vertebral fragments associated with a pars fracture as well as surrounding vertebrae, in order to provide additional fixation during the healing process of the fracture, among other things.

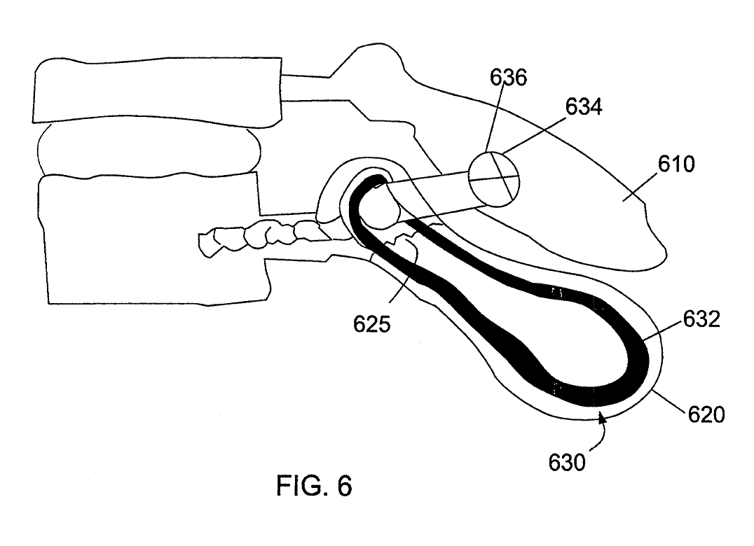

FIG. 6 depicts a view 600 of a bone plate 630 that provides both fixation of vertebral fragments and attachment between an affected vertebra and surround vertebrae. The bone plate 630 includes a fragment fixation section 632 and a support section 634. The bone plate 630 utilizes the fragment fixation section 632, as described herein, to rigidly fix vertebral fragments of a vertebra 620 that contains a fracture 625. Furthermore, the bone plate 630 includes a support section 634 that fixes the affected vertebra 620 to a surrounding vertebra 610 via the bone plate, providing bilateral support to the affected vertebra while rigidly fixing the vertebral fragments, among other things.

The support section 634 may include an attachment portion 636 that attaches the bone plate 630, via the support section 634 to surrounding vertebrae. For example, the attachment portion 636 may include an opening that enables a screw to attach the support section 634 to vertebra 610, may include a wire or wiring mechanism that attaches the support section 634 to vertebra 610, may include a portion that facilitates fusion of the vertebra 610 to the bone plate 630 or to affected vertebra 620, and so on.

Thus, in certain embodiments, the bone plate 600 furthers and expands upon the state of the art by providing a device that both rigidly fixes vertebral fragments together, improving over the conventional practice of using wires, and attaches an affected vertebra to surrounding vertebrae, improving over the conventional practice of fusing vertebrae together.

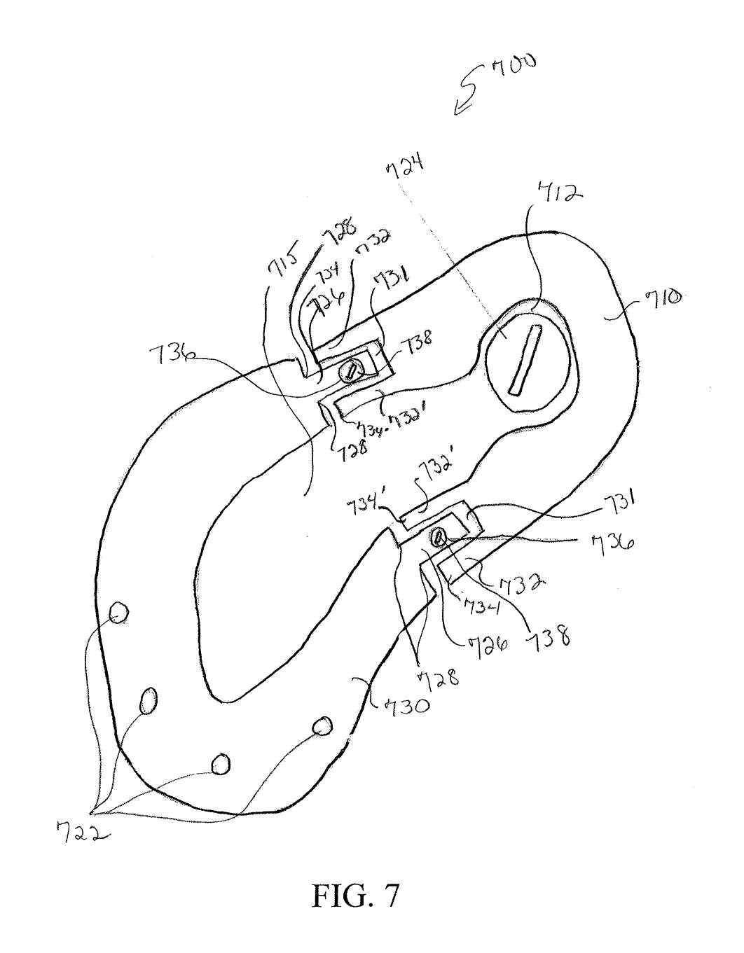

FIG. 7 is a perspective view of a two-piece peripheral rim bone plate 700 similar to the embodiments disclosed herein that includes a lamina plate section 730 that is configured to interlock with a pedicle plate section 710. Bone plate 700 is configured to attach to a pars area of a vertebra. Bone plate 700 is used for rigid fixation of a fracture in the pars area in order to rigidly fix a pedicle to a lamina in order to facilitate successful healing of the fracture by allowing access to the fracture during the healing process.

The bone plate 700 includes a pedicle section 710 that is of a shape similar to the shape of a pedicle. The pedicle section 710 of the bone plate 700 has an outer rim configured to conform to the outer edge of a pedicle and an inner rim that defines an opening 712 that enables a pedicle screw 724 to fix the pedicle section 710 of the bone plate 700 to a pedicle. Opening 712 is in fluid communication with lamina opening 715.

As shown, the outer rim of pedicle section 710 is shaped and/or is formed having a geometry that is similar to a shape and/or anatomical geometry of an outer edge of a pedicle. Shaping the pedicle section 710 in such a manner enables the bone plate 700 to conform to the area in which it is fixed, which allows for the bone plate to be placed within such a dense, complex area of a spinal column, among other benefits.

The bone plate 700 also includes a lamina section 730 having an outer rim that conforms to the outer edge or anatomical geometry of a lamina. The lamina section 730 of the bone plate 700 includes one or more lamina screw receiving openings 722 that facilitate attachment of the bone plate 700 to the lamina. Pedicle section 710 defines a pedicle screw receiving opening 712 for receiving a pedicle screw.

As shown, the outer rim of the lamina section 730 of the bone plate 700 is shaped and/or is formed having a geometry that is similar to a shape and/or geometry of an outer edge of the lamina 360 enabling the bone plate 700 to conform to the area in which it is fixed, which allows for the bone plate to be placed within such a dense, complex area of a spinal column, among other benefits.

The lamina section 730 includes projection 726 extending radially from step 728 formed in lamina section 730. Pedicle section 710 includes an opening 730 defined by opposing arms 732, 732′ having surfaces 734, 734′. Arms 732, 734′ include a radially extending portion 735 that mates with receiving channel 737 on projection 726. Those of skill in the art will appreciate that the extending portion 735 may be on projection 726 while the mating receiving channel 737 may be on arms 732, 734. Projection 726 including a screw opening 736 therethrough for receiving a screw 738. In operation opening 731 receives projection 728 such that surfaces 734, 734′ abut step 728. As the screw is tightened the pedicle plate section 710 compress to the lamina section 730 to form a unitary structure. Those of skill in the art will appreciate that projection 726 and opening 731 are depicted as having rectangular shapes but any interlocking shape or geometry such as round, dove-tail, angular, zig-zag and the like may be utilized and intended to fall within the scope of the invention. Those of skill in the art will also appreciate that projection 726 may be on the pedicle plate portion 710 while the receiving opening 731 may be on the lamina plate portion 730.

Bone plate 700 includes an inner open portion 715 that provides access to the fracture within the pars area of the vertebra. That is, the bone plate 700 may be configured to give surgeons access to the fracture in order to perform bone grafting procedures while the bone plate 710 rigidly maintains the vertebral fragments together. Inner open portion 715 may be configured to allow for local vascular in-growth of the fracture and may be configured to enable access of the fracture for post operation CT Scan imaging procedures, and so on.

As will be appreciated by those of skill in the art, inner open portion 715 is in fluid communication with, contiguous with and overlaps with the pedicle screw receiving opening 712.

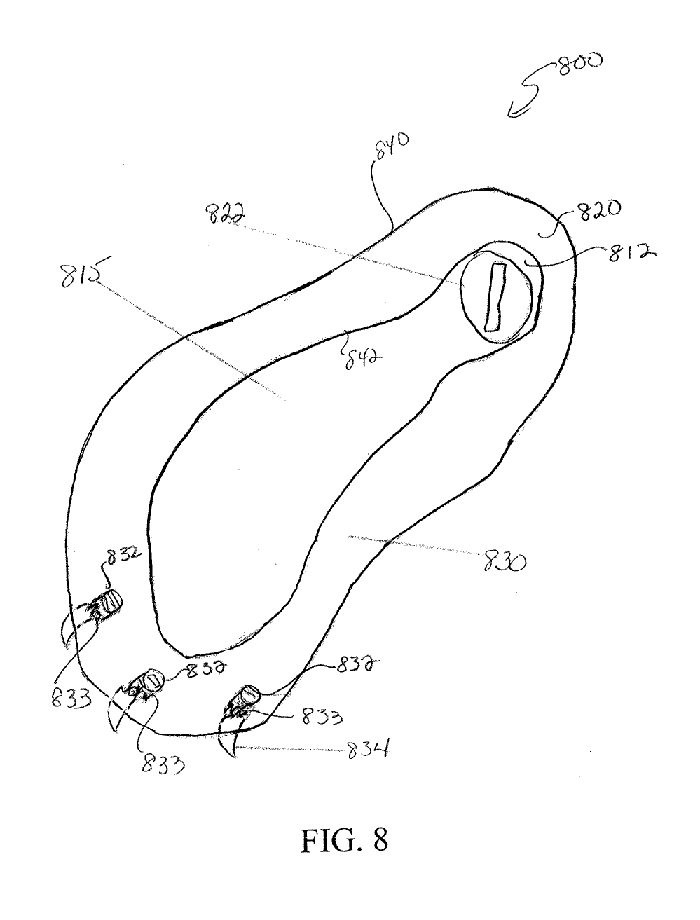

FIG. 8 is a perspective view of a peripheral rim bone plate 800 that includes an opening on the lamina portion for receiving a crankshaft screw therethrough. The crankshaft screw is configured to advance a hook into a lamina to secure the bone plate to the lamina. Those of skill in the art will appreciate that a crankshaft screw may be utilized with any of the various embodiments of the bone plate as depicted in FIGS. 2-3 and 5-8. As depicted, bone plate 800 includes a pedicle section 820 that is of a shape similar to the shape of a pedicle. The pedicle section 820 of the bone plate 800 defines a screw receiving opening 812 that enables a screw 822 to fix the pedicle section 820 of the bone plate 800 to a pedicle.

As shown, the pedicle section 820 of the bone plate 800 is shaped and/or is formed having a geometry that is similar to a shape and/or anatomical geometry of a pedicle. Shaping the pedicle section 820 in such a manner enables the bone plate 800 to conform to the area in which it is fixed, which allows for the bone plate 800, as with other embodiments disclosed herein, to be placed within such a dense, complex area of a spinal column, among other benefits.

The bone plate 800 also includes a lamina section 830 that is of a shape similar to a shape and/or anatomical geometry of a lamina. The lamina section 830 of the bone plate 800 includes openings 832 therethrough for receiving a crankshaft screw 833 therethrough by rotatably advance a hook 834 into the lamina to attach the bone plate 800 to the lamina.

As shown, the lamina section 830 of the bone plate 800 is shaped and/or is formed having a geometry that is similar to a shape and/or geometry of a lamina and/or spinous process. Shaping the lamina section 830 in such a manner enables the bone plate 800 to conform to the area in which it is fixed, which allows for the bone plate to be placed within such a dense, complex area of a spinal column, among other benefits.

In certain embodiments, the bone plate 800 includes an inner open portion 815 that provides access to a fracture within the pars area of a vertebra. That is, the bone plate 800 may be configured to give surgeons access to a fracture in order to perform bone grafting procedures while the bone plate 800 rigidly maintains the vertebral fragments together, may be configured to allow for local vascular in-growth of the fracture, may be configured to enable access of the fracture 870 for post operation CT Scan imaging procedures, and so on.

Thus, in certain embodiments, the bone plate 800 includes an outer peripheral rim 840 that is shaped similar to areas of a vertebra in which the bone plate attaches and is shaped similar to the areas of the vertebra in which the bone plate attaches, and an inner rim 842 that defines the inner open portion 815 that enables access to a fractured or repaired area of the vertebra. Inner rim 842 of the pedicle section 820 defines the pedicle screw receiving opening 812. As will be appreciated by those of skill in the art, inner open portion 815 is in fluid communication with, contiguous with and overlaps with the pedicle screw receiving opening 812.

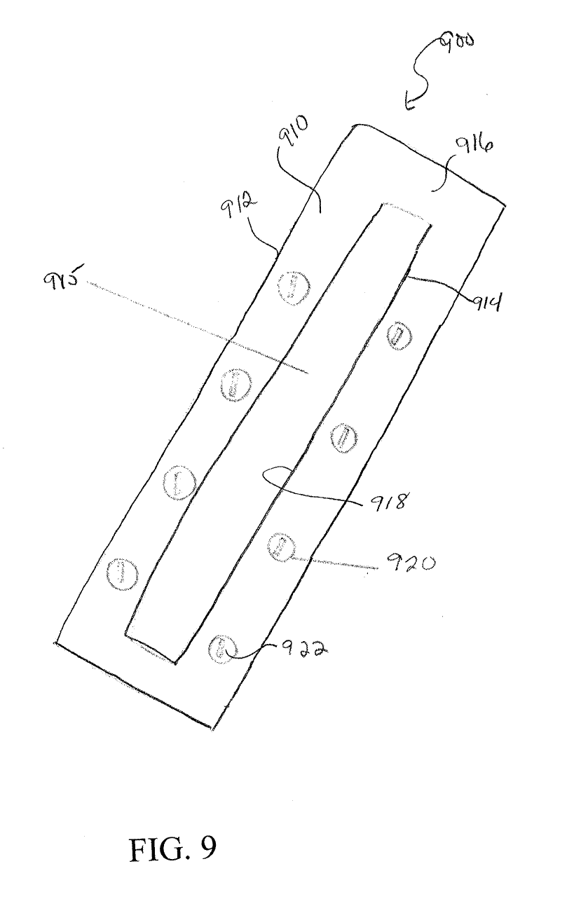

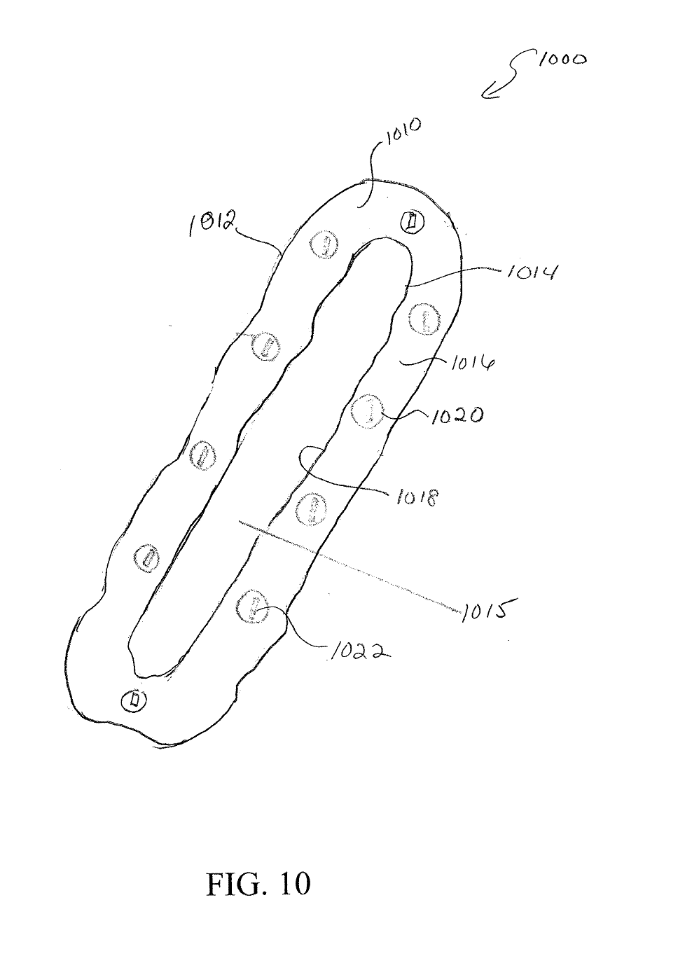

FIGS. 9 and 10 are perspective views of a bone plate 900, 1000 that may be utilized to rigidly fix fragments of a long bone to one another. Long bones function to support the weight of the body and facilitate movement. Long bones are mostly located in the appendicular skeleton and include bones in the lower limbs (the tibia, fibula, femur, metatarsals, and phalanges) and bones in the upper limbs (the humerus, radius, ulna, metacarpals, and phalanges). Long bones are typically cylindrical in shape. FIG. 9 depicts a regularly shaped bone plate 900 while FIG. 10 depicts a bone plate that is irregularly shaped and configured to conform to the geometry of the particular long bone that is fractured.

Bone plate 900 includes an elongate, rectangular shaped, peripheral rim 910. Peripheral rim includes an outer regularly shaped edge 912 and an inner regularly shaped edge 914. Inner edge 914 defines open area 915. Elongate peripheral rim 910 includes an upper surface 916 and a lower bone-contacting surface 918. One or more screw receiving holes 920 are positioned through upper surface 916 and lower bone contacting surface 918 for receiving screws 922 therethrough. Screws 922 secure elongate peripheral rim 910 to a long bone. Screws 922 may comprise crankshaft screws that rotatably secure a hook into a long bone. Lower bone contacting surface 918 may conform to a two- or three-dimensional geometry of the long bone to which it is fixed. Advantageously, bone plate 900 is placed over a fracture in a long bone and open area 915 is configured to give surgeons access to a fracture in a long bone in order to perform bone grafting procedures while the bone plate 900 rigidly maintains the vertebral fragments together, may be configured to allow for local vascular in-growth of the fracture, may be configured to enable access of the fracture for post operation CT Scan imaging procedures, and so on.

FIG. 10 depicts a bone plate 1000 similar to the bone plate of FIG. 9. Bone plate 1000 includes an elongate, rectangular shaped, peripheral rim 1010. Peripheral rim 1010 includes an outer irregularly shaped edge 1012 and an inner irregularly shaped edge 1014. Outer and inner edges 912, 914 are configured to conform to the geometry of the particular long bone that it is rigidly fixed thereto. Inner edge 1014 defines open area 1015. Elongate peripheral rim 1010 includes an upper surface 1016 and a lower bone-contacting surface 1018. Lower surface 1018 is configured to conform to a two- or three-dimensional geometry of the long bone to which it is affixed and prepared in accordance with the process depicted in FIG. 4 and disclosed herein. One or more screw receiving holes 1020 are positioned through upper surface 1016 and lower surface 1018 for receiving screws 1022 therethrough. Screws 1022 secure elongate peripheral rim 1010 to a long bone. Screws 1022 may comprise crankshaft screws that rotatably secure a hook into a long bone. Advantageously, bone plate 1000 is placed over a fracture in a long bone and open area 1015 is configured to give surgeons access to a fracture in a long bone in order to perform bone grafting procedures while the bone plate 1000 rigidly maintains the vertebral fragments together, may be configured to allow for local vascular in-growth of the fracture, may be configured to enable access of the fracture for post operation CT Scan imaging procedures, and so on.

Various modifications and additions can be made to the embodiments disclosed herein without departing from the scope of the present invention. For example, while the embodiments described above refer to particular features and aspects, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed:

1. A two-piece bone plate configured to rigidly fix a single pedicle to a single lamina, the bone plate comprising a first elongate section configured to be fixed to a pedicle, said first section having an outer rim configured to conform to an outer edge of a pedicle and an inner rim defining a pedicle screw receiving hole; and a second elongate section configured to be fixed to a lamina, said second elongate section having an outer rim configured to conform to an outer edge of a lamina and an inner rim defining a lamina open area, said lamina open area in fluid communication with said pedicle screw receiving hole, said first elongate section and said second elongate section including interlocking portions thereon configured to matingly engage said first elongate section to said second elongate section.

2. The two-piece bone plate of claim 1, further comprising one or more screw holes extending through said second elongate section configured to receive one or more lamina screws to fix the second elongate plate section to the single lamina.

3. The two-piece bone plate of claim 1, wherein said interlocking portions include a projection radially extending from the second elongate plate section and a receiving opening on the first elongate plate section for receiving the projection.

4. The bone plate of claim 1, wherein said interlocking portions include a projection radially extending from the first plate section and a receiving opening on the second elongate plate section for receiving the projection.

5. The bone plate of claim 1, wherein the interlocking portions are selected from rectangular, round, zig-zag and dove-tail shapes.

6. A bone plate configured to rigidly fix fragments of a long bone to one another, the bone plate comprising an elongate peripheral rim having an outer rim configured to conform to an anatomical contour of an outer edge of the long bone and an inner rim defining an open area; the bone plate including an upper surface and a lower bone-contacting surface, said lower bone contacting surface configured to conform to a two- or three-dimensional geometry of the long bone; and one or more openings extending through said upper surface through said lower bone-contacting surface for receiving one or more bone fastening devices therethrough, wherein said open area is configured to provide access to the fracture during a bone healing process.
